(19) United States
(12) Patent Application Publication
(10) Pub. No.: US 2026/0347777 A1
(43) Pub. Date: Nov. 5, 2026

(54) TRANSFORMED CELL FOR PRODUCING RED BLOOD CELLS, AND USE THEREOF

(71) Applicant: REDGENE INC., lksan-si, Jeonbuk-do (KR)

(72) Inventors: Kaapjoo PARK, lksan-si (KR); Omer HABIB, lksan-si (KR); Gizem HABIB, lksan-si (KR); Dong Hee KIM, lksan-si (KR); Hee Yeon CHO, lksan-si (KR); Younsu LEE, lksan-si (KR)

(73) Assignee: REDGENE INC., lksan-si, Jeonbuk-do (KR)

(21) Appl. No.: 19/484,621

(22) PCT Filed: May 20, 2024
(86) PCT No.: PCT/KR2024/006793

§ 371 Date: Nov. 14, 2025

(30) Foreign Application Priority Data
May 18, 2023 (KR) 10-2023-0064132
Jan. 25, 2024 (KR) 10-2024-0011571

(51) Int. Cl. C12N 5/078; C07K 14/505; C07K 14/705; C12N 15/85

(52) U.S. Cl. CPC C12N 5/0641; C07K 14/505; C07K 14/70517; C12N 15/85; C07K 2319/60; C12N 2501/14; C12N 2502/45; C12N 2502/99

(57) ABSTRACT

The present disclosure relates to a transformed cell for producing red blood cells, and more specifically: to the transformed cell for producing red blood cells; and a method for producing and differentiating red blood cells using the same. Applying the transformed cells for producing red blood cells according to the present disclosure to the differentiation and production of in vitro red blood cells was found to significantly reduce production costs compared to conventional methods for differentiating and producing red blood cells by adding erythropoietin, and enable the mass production of red blood cells. Thus, the transformed cell for producing red blood cells according to the present disclosure can be variously used in clinical fields related to blood, as well as the field of treating diseases related to red blood cell reduction.

### TECHNICAL FIELD

The present disclosure relates to a transformed cell for producing red blood cells, and more particularly, to a transformed cell for producing red blood cells; and a method for producing and differentiating red blood cells using the same.

### BACKGROUND ART

Red blood cells as a component of blood play an important role in transporting oxygen to human tissues and carbon dioxide in the tissues to the outside of the body. In particular, a cause of shock during excessive bleeding is a lack of red blood cells. However, until now, the only available method for replacing red blood cells is transfusing blood from another person. Recently, a current system is unable to provide a smooth supply of red blood cells due to a shortage of blood donations caused by the recent global pandemic and aging population, the risk of transmission of infectious diseases through transfusions, etc. As alternatives, there have been attempts to develop hemoglobin solutions or oxygen carriers to replace red blood cells, but their clinical application has been prohibited due to serious side effects. Accordingly, research has been actively conducted to produce red blood cells in vitro.

Stem cells are attracting attention for the in vitro production of red blood cells. In particular, hematopoietic stem cells are produced in the bone marrow and are mother cells of red blood cells and white blood cells. The red blood cells are produced from hematopoietic stem cells through several differentiation stages, in which nuclei are gradually condensed and the cell sizes are decreased through pronormoblasts, basophilic erythroblasts, polychromatic erythroblasts, and orthochromatic erythroblasts. In addition, the condensed nuclei are enucleated, and then become reticulocytes and matured into erythrocytes. This differentiation process is typically performed over three weeks in vitro, and erythropoietin (EPO) is an essential culture additive for erythroid differentiation through the entire differentiation process.

The erythropoietin is a glycoprotein hormone that is involved in production of red blood cells, and is produced in the in vivo kidneys and even in the liver during the fetal period. The erythropoietin is an essential growth factor for mature erythroid differentiation by acting on erythroid progenitor cells to stimulate cell proliferation and differentiation.

### DISCLOSURE

### Technical Problem

Accordingly, the present inventors developed an MB-EPO cell line expressing inducible erythropoietin for in vitro production of red blood cells, and confirmed that red blood cells were able to be produced without adding EPO in the case of using the MB-EPO cell line, and then completed the present disclosure.

Therefore, an object of the present disclosure is to provide an expression cassette for producing red blood cells, including an erythropoietin (EPO) gene represented by a nucleotide sequence of SEQ ID NO: 12; and a gene encoding a transmembrane domain.

Another object of the present disclosure is to provide a recombinant vector for producing red blood cells including an expression cassette for producing red blood cells including an erythropoietin (EPO) gene represented by a nucleotide sequence of SEQ ID NO: 12; and a gene encoding a transmembrane domain.

Yet another object of the present disclosure is to provide a transformed cell for producing red blood cells, which is introduced with an expression cassette for producing red blood cells including an erythropoietin (EPO) gene represented by a nucleotide sequence of SEQ ID NO: 12; and a gene encoding a transmembrane domain; or a recombinant vector including the expression cassette.

Still another object of the present disclosure is to provide a composition for producing red blood cells including the expression cassette, the recombinant vector or the transformed cell.

Still another object of the present disclosure is to provide a kit for producing red blood cells including: a) a first compartment including a recombinant vector for producing red blood cells; and b) a second compartment including an expression inducer.

Still another object of the present disclosure is to provide a method for producing an erythropoietin receptor-activating cell, including transforming the expression cassette or the recombinant vector into a stem cell.

Still another object of the present disclosure is to provide a method for inducing in vitro erythroid differentiation, including an erythroid differentiation step of culturing a transformed cell and an erythroid progenitor cell for producing red blood cells into which the expression cassette or the recombinant vector has been introduced.

### Technical Solution

In order to achieve the object, an aspect of the present disclosure provides an expression cassette for producing red blood cells, including an erythropoietin (EPO) gene represented by a nucleotide sequence of SEQ ID NO: 12; and a gene encoding a transmembrane domain.

Another aspect of the present disclosure provides a recombinant vector for producing red blood cells including an expression cassette for producing red blood cells including an erythropoietin (EPO) gene represented by a nucleotide sequence of SEQ ID NO: 12; and a gene encoding a transmembrane domain.

Yet another aspect of the present disclosure provides a transformed cell for producing red blood cells, which is introduced with an expression cassette for producing red blood cells including an erythropoietin (EPO) gene represented by a nucleotide sequence of SEQ ID NO: 12; and a gene encoding a transmembrane domain; or a recombinant vector including the expression cassette.

Still another aspect of the present disclosure provides a composition for producing red blood cells including the expression cassette, the expression cassette, the recombinant vector or the transformed cell.

Still another aspect of the present disclosure provides a kit for producing red blood cells including: a) a first compartment including a recombinant vector for producing red blood cells; and b) a second compartment including an expression inducer.

Still another aspect of the present disclosure provides a method for producing an erythropoietin receptor-activating cell, including transforming the expression cassette or the recombinant vector into a stem cell.

Still another aspect of the present disclosure provides a method for inducing in vitro erythroid differentiation, including an erythroid differentiation step of culturing a transformed cell and an erythroid progenitor cell for producing red blood cells into which the expression cassette or the recombinant vector has been introduced.

### Advantageous Effects

According to the present disclosure, by applying the transformed cells for producing red blood cells to in vitro differentiation and production of red blood cells, it can be confirmed that not only production costs are very low compared to conventional methods for differentiating and producing red blood cells by adding erythropoietin, but also red blood cells can be mass-produced. Therefore, the transformed cell for producing red blood cells of the present disclosure can be variously used in clinical fields related to blood and the field of treating diseases related to red blood cell reduction.

### DESCRIPTION OF DRAWINGS

FIG. 1A is a diagram showing an inactive state of an EPO receptor.

FIG. 1B is a diagram showing a mechanism of action of inducible EPO in an MB-EPO cell according to the present disclosure.

FIGS. 2A and 2B are diagrams showing a cleavage map of a recombinant vector for constructing an MB-EPO cell according to the present disclosure.

FIG. 3A is a diagram showing results of observing UT7 cells cultured in the presence and absence of EPO.

FIG. 3B is a diagram showing results of analyzing cell growth of UT cells depending on the presence of EPO.

FIG. 4A is a diagram showing results of analyzing cell growth of UT7-MB-EPO cells in the presence of EPO.

FIG. 4B is a diagram showing results of analyzing cell growth of UT7-MB-EPO cells in the absence of EPO.

FIG. 5A is a schematic diagram showing cell differentiation of a UT7 cell into which a GFP gene is introduced in the absence of EPO.

FIG. 5B is a schematic diagram showing cell differentiation of a UT7-MB-EPO cell into which MB-EPO is introduced in the absence of EPO.

FIG. 6A is a diagram showing a TALEN-mediated targeting strategy of an AAVS1 locus for constructing MB-EPO-KI hiPSCs.

FIG. 6B is a diagram showing results of confirming the expression of doxycycline-dependent MB-EPO in MB-EPO-KI hiPSCs through flow cytometry.

FIG. 7 is a schematic diagram showing an in vitro red blood cell producing process using hiPSCs; and a designed experimental group.

FIG. 8A is a diagram showing results of observing the cell morphology of a control group CMC11 WT during mesoderm induction.

FIG. 8B is a diagram showing results of observing the cell morphology of MB-EPO-KI hiPSCs during mesoderm induction.

FIG. 9 is a diagram showing results of observing the cell morphology of a control group WT and MB-EPO-hiPSCs according to the presence of EPO and doxycycline during endothelial-to-hematopoietic transition.

FIG. 10A is a diagram showing results of evaluating erythroid differentiation of a control group CMC11 WT according to the presence of EPO and doxycycline after induction of erythroid differentiation.

FIG. 10B is a diagram showing results of evaluating erythroid differentiation of MB-EPO-KI hiPSCs according to the presence of EPO and doxycycline after induction of erythroid differentiation.

### BEST MODE

Hereinafter, the present disclosure will be described in detail.

According to an aspect of the present disclosure, the present disclosure provides an expression cassette for producing red blood cells, including an erythropoietin (EPO) gene represented by a nucleotide sequence of SEQ ID NO: 12; and a gene encoding a transmembrane domain.

In the present disclosure, the expression cassette refers to a unit cassette that includes a promoter and a gene encoding a target protein and may be expressed to produce the target protein operably linked to the downstream of the promoter. Various factors capable of helping the efficient production of the target protein may be included inside or outside such an expression cassette. Specifically, in the target protein expression cassette, the gene encoding the target protein may be operably linked to the downstream of the promoter sequence.

The ‘operably linked’ means that the gene sequence and the promoter sequence are functionally linked to each other so that a nucleic acid sequence having the promoter activity of the present disclosure initiates and mediates the transcription of the gene encoding the target protein. The operable linkage may be prepared using genetic recombination techniques known in the art, and site-specific DNA cleavage and linkage may be prepared using cleavage and linkage enzymes in the art, but are not limited thereto.

In addition, the ‘gene expression cassette’ may be inserted into a chromosome of a host cell to be used for preparing a recombinant microorganism, and it is obvious to those skilled in the art to which the present disclosure pertains that the same effect is achieved as a case of introducing the recombinant vector into the host cell as described above even though the recombinant gene expression cassette is inserted into the genomic chromosome of a host cell.

As a method of inserting the recombinant gene expression cassette into the chromosome of the host cell, conventionally known genetic engineering methods may be used. As an example, the method includes methods using a retrovirus vector, an adenovirus vector, an adeno-associated virus vector, a herpes simplex virus vector, a poxvirus vector, a lentiviral vector, or a non-viral vector.

In a specific embodiment of the present disclosure, it is preferable that the transmembrane domain is at least one selected from the group consisting of a receptor, a ligand, an immunoglobulin, and a glycophorin. Specific examples of the transmembrane domain may include at least one selected from the group consisting of CD8, CD4, CD3, CD3y, CD38, CD3S, CD28, CD137, Fcely, T cell receptor, nicotinic acetylcholine receptor, and gamma-aminobutyric acid receptor, and most preferably, a cell surface antigen CD8 represented by a nucleotide sequence of SEQ ID NO: 15. In the present disclosure, the cell surface antigen CD8 is a transmembrane glycoprotein that serves as a co-receptor of the T cell receptor. In particular, the cell surface antigen CD8 is expressed on almost all cells of bone marrow-derived cells. Accordingly, in the embodiment of the present disclosure, the transmembrane domain was designed so that EPO may continuously affect cells even when a culture medium is replaced, by binding self-secreted EPO to the cell surface antigen CD8.

In a specific embodiment of the present disclosure, the expression cassette may further include a linker, and preferably, the linker is encoded by a nucleotide sequence of SEQ ID NO: 13.

In a specific embodiment of the present disclosure, the expression cassette may further include a reporter gene, and the reporter gene may be a gene encoding a fluorescent protein. The fluorescent protein is not particularly limited in type as long as the fluorescent protein is any protein known in the art which exhibits fluorescence, but may be used with mCherry, green fluorescent protein (GFP), modified green fluorescent protein, enhanced green fluorescent protein (EGFP), red fluorescent protein (RFP), enhanced red fluorescent protein (ERFP), blue fluorescent protein (BFP), enhanced blue fluorescent protein (EBFP), yellow fluorescent protein (YFP), enhanced yellow fluorescent protein (EYFP), cyan fluorescent protein (CFP), or enhanced cyan fluorescent protein (ECFP), and preferably, mCherry or green fluorescent protein (GFP).

In a specific embodiment of the present disclosure, the expression cassette and the recombinant vector for producing red blood cells including the expression cassette may further include a gene capable of inducing the expression of a target protein by an expression inducer, and examples of the gene include a promoter expressed by the expression inducer, a transcription activator, etc. The expression inducer may be doxycycline or tetracycline.

In a specific embodiment of the present disclosure, it is preferable that in the expression cassette, an erythropoietin gene, a linker, and a gene encoding a transmembrane domain are sequentially operably linked. In an embodiment of the present disclosure, an expression cassette for producing red blood cells was constructed in which an erythropoietin gene (SEQ ID NO: 12), a linker (SEQ ID NO: 13), a CD8 gene (SEQ ID NO: 15), and an mCherry gene (SEQ ID NO: 19) were sequentially and operably linked, which was named ‘MB-EPO’ by the present inventors. The constructed expression cassette MB-EPO is represented by a nucleotide sequence of SEQ ID NO: 11 and encodes a polypeptide represented by an amino acid sequence of SEQ ID NO: 1.

In addition, variants of the nucleotide sequences are included within the scope of the present disclosure. Specifically, the gene has a sequence homology of 70% or more, more preferably 80% or more, even more preferably 90% or more, and most preferably 95% or more with the nucleotide sequences of the erythropoietin gene, the linker, and the gene encoding the transmembrane domain, and means a sequence that exhibits substantially the same physiological activity. The “% of sequence homology” with the polynucleotide is confirmed by comparing two optimally aligned sequences with a comparison region, and a part of the polynucleotide sequence in the comparison region may include addition or deletion (i.e., gap) compared to a reference sequence (without addition or deletion) for an optimal alignment of the two sequences.

According to another aspect of the present disclosure, the present disclosure provides a recombinant vector for producing red blood cells including an expression cassette for producing red blood cells including an erythropoietin (EPO) gene represented by a nucleotide sequence of SEQ ID NO: 12; and a gene encoding a transmembrane domain.

In the present disclosure, the vector refers to a genetic construct including a nucleotide sequence of a gene operably linked to a suitable regulatory sequence so as to express a target gene in a suitable host. The regulatory sequence may include a promoter capable of initiating transcription, any operator sequence for regulating such transcription, and sequences for regulating termination of transcription and translation.

In the present disclosure, when an encoding gene of a target polypeptide to be expressed is operably linked, the recombinant vector may be used as an expression vector of a target polypeptide capable of expressing the target polypeptide with high efficiency in a suitable host cell, and the recombinant vector can be expressed in a host cell. The host cell may preferably be a eukaryotic cell, and expression regulatory sequences such as a promoter, a terminator, and an enhancer, sequences for membrane targeting or secretion, and the like are appropriately selected according to a type of host cell and may be variously combined depending on a purpose.

For example, the vector of the present disclosure includes plasmid vectors, cosmid vectors, and viral vectors such as a bacteriophage vector, an adenovirus vector, a retrovirus vector, and an adeno-associated virus vector. The vector which may be used as the recombinant vector may be prepared by manipulating plasmids (e.g., pGLS, pSC101, pGV1106, pACYC177, ColE1, pKT230, ME290, pBR322, pUC8/9, pUC6, pBD9, pHC79, pIJ61, pLAFR1, pHV14, pGEX series, pET series, pUC19, etc.), phages (e.g., λgt4λB, λCharon, λΔz1, M13, etc.) or viruses (e.g., CMV, SV40, etc.), which have often been used in the art.

In the recombinant vector, polynucleotides encoding CYP3A4, CYP1A2, CYP2B6, CYP2D6, CYP2C9, CYP2C19, CYP2E1 and CYP2A6 may be operably linked to a promoter. The term “operatively linked” refers to a functional linkage between a nucleotide expression regulatory sequence (e.g., a promoter sequence) and another nucleotide sequence. Accordingly, the regulatory sequence may regulate transcription and/or translation of another nucleotide sequence.

The recombinant vector may typically be constructed as a vector for cloning or a vector for expression. As the expression vector, conventional vectors used in the art to express foreign proteins in plants, animals, or microorganisms may be used. The recombinant vector may be constructed through various methods known in the art.

The recombinant vector may be constructed using a prokaryotic or eukaryotic cell as a host. For example, in the case of using the eukaryotic cell as the host, a replication origin operating in the eukaryotic cell included in the vector includes an f1 replication origin, an SV40 replication origin, a pMB1 replication origin, an adeno replication origin, an AAV replication origin, a CMV replication origin, and a BBV replication origin, but is not limited thereto. In addition, a promoter (e.g., metallothionein promoter) derived from a genome of a mammalian cell or a promoter (e.g., an adenovirus late promoter, a vaccinia virus 7.5K promoter, a SV40 promoter, a cytomegalovirus (CMV) promoter and a tk promoter of HSV) derived from a mammalian virus may be used, and a polyadenylation sequence is generally included as a transcription termination sequence.

In a specific embodiment of the present disclosure, the recombinant vector may include a promoter that regulates the expression of a gene, and more preferably, the promoter is a tetracycline response element (TRE) promoter, but the scope of the present disclosure is not limited thereto. The promoter that regulates the expression of the gene of the present disclosure may initiate gene expression by an expression inducer to regulate the expression of the target protein according to a purpose. The expression inducer may be doxycycline or tetracycline.

According to yet another aspect of the present disclosure, the present disclosure provides a transformed cell for producing red blood cells, which is introduced with an expression cassette for producing red blood cells including an erythropoietin (EPO) gene represented by a nucleotide sequence of SEQ ID NO: 12; and a gene encoding a transmembrane domain; or a recombinant vector including the expression cassette.

The host cell may also be used with any host cell known in the art, and in the case of transformation to eukaryotic cells, yeast (Saccharomyces cerevisiae), insect cells, plant cells, and animal cells, for example, SP2/0, Chinese hamster ovary (CHO) K1, CHO DG44, PER.C6, W138, BHK, COS-7, 293, HepG2, Huh7, 3T3, RIN, and MDCK cell lines may be used as the host cell.

In a specific embodiment of the present disclosure, the transformed cell is preferably a stem cell, and more preferably, at least one selected from the group consisting of an induced pluripotent stem cell (iPSC), a hematopoietic stem cell (HSC), and a mesenchymal stem cell (MSC).

The pluripotent stem cell may include, without limitation, stem cells separated or isolated from a patient or donor, or commercially available stem cell lines. The pluripotent stem cell is also commonly known as a PS cell, and includes any cell capable of differentiating into almost all cells, that is, cells derived from any of three germ layers (germinal epithelium), including endoderm (inner stomach wall, gastrointestinal tract, lung), mesoderm (muscle, bone, blood, urogenital organ), and ectoderm (epithelial tissues and nervous system). The PSC may be derived from embryonic stem cells (including embryonic germ cells) or may be the descendants of totipotent cells obtained by inducing non-pluripotent cells, such as adult somatic cells, by forcing the expression of a specific gene.

In addition, the induced pluripotent stem cell (iPSC) is also commonly abbreviated as an iPS cell, and refers to a type of pluripotent stem cell artificially induced from a non-pluripotent cell, such as an adult somatic cell normally by inducing the “forced” expression of a specific gene.

The origin of the stem cells is human and non-human animals (e.g., mouse, rat, cow, horse, pig, sheep, monkey, dog, cat, etc.), and is not particularly limited, but is particularly preferably a human-derived cell.

The insertion of the recombinant vector into the host cell may be performed using an insertion method widely known in the art. When the host cell is a eukaryotic cell, the delivery method may be used with microinjection, calcium phosphate precipitation, electroporation, liposome-mediated transfection, gene bombardment, etc., but is not limited thereto.

The method for selecting the transformed host cell may be easily performed using a phenotype expressed by a selection marker, according to a method widely known in the art. For example, when the selection marker is a specific antibiotic-resistant gene, a transformant may be easily selected by culturing the transformant in a medium containing the antibiotic.

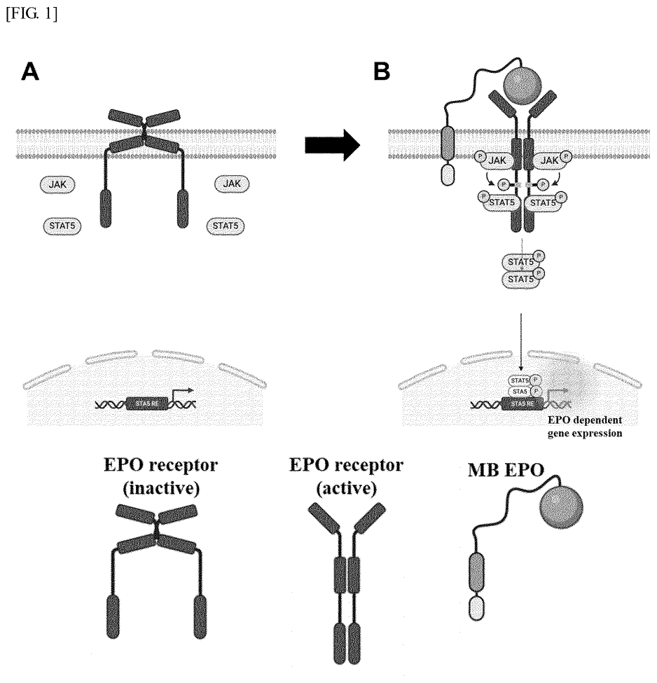

In a specific embodiment of the present disclosure, the transformed cell is preferably bound to a transmembrane domain, and more preferably, the transmembrane domain may be bound to a cell membrane. That is, in the transformed cell, MB-EPO is bound to the cell membrane as shown in FIG. 1B, and the MB-EPO bound to the cell membrane continuously affects the EPO receptor of the cell, thereby continuously inducing the expression of an EPO-dependent gene.

According to yet another aspect of the present disclosure, the present disclosure provides a composition for producing red blood cells including the expression cassette, the recombinant vector or the transformed cell.

The composition for producing red blood cells of the present disclosure may further include known substances required for maintenance, growth, proliferation, differentiation induction, etc. of constituent components.

The composition for producing red blood cells according to the present disclosure may induce differentiation and produce red blood cells without addition of erythropoietin, and thus can be effectively used in clinical fields related to blood; and in the field of treating diseases related to red blood cell reduction.

According to yet another aspect of the present disclosure, the present disclosure provides a kit for producing red blood cells including: a) a first compartment including a recombinant vector for producing red blood cells; and b) a second compartment including an expression inducer.

The recombinant vector for producing red blood cells includes an expression cassette for producing red blood cells, including an erythropoietin (EPO) gene represented by a nucleotide sequence of SEQ ID NO: 12; and a CD8 gene represented by a nucleotide sequence of SEQ ID NO: 15. Preferably, the recombinant vector includes a promoter that regulates the expression of a gene, and the promoter may be a tetracycline response element (TRE) promoter.

The first compartment of the kit for producing red blood cells of the present disclosure may further include additional components for maintaining and preserving the expression cassette, the recombinant vector or the transformed cell. In addition, the second compartment of the present disclosure may further include a component for maintaining and preserving the expression inducer.

The kit for producing red blood cells of the present disclosure may additionally include reagents, articles, etc. known in the art used for producing red blood cells. In addition, the kit for producing red blood cells of the present disclosure may additionally include user instructions describing optimal performance conditions. The instructions describe a method for using a kit, for example, reaction conditions to be presented, the order of each step, etc. The instructions include pamphlets or leaflet-type brochures, labels attached to the kit, and instructions on the surface of a package including the kit. In addition, the instructions may include information published or provided through electronic media such as the Internet.

In a specific embodiment of the present disclosure, the expression inducer may be doxycycline or tetracycline.

According to yet another aspect of the present disclosure, the present disclosure provides a method for producing erythropoietin receptor-activating cells, including transforming the expression cassette or the recombinant vector into a stem cell.

In a specific embodiment of the present disclosure, the erythropoietin receptor-activating cell may express erythropoietin bound to a transmembrane domain located in the cell membrane.

In a specific embodiment of the present disclosure, the expression cassette or the recombinant vector may include a promoter that regulates the expression of a gene.

Since the expressed erythropoietin is bound to the cell surface antigen CD8 as shown in FIG. 1B, the erythropoietin receptor-activating cell produced by the method of the present disclosure may continuously activate an erythropoietin receptor, and can induce the expression according to the purpose by the promoter that regulates gene expression, and the expression inducer.

According to yet another aspect of the present disclosure, the present disclosure provides a method of inducing in vitro erythroid differentiation, including an erythroid differentiation step of culturing a transformed cell and an erythroid progenitor cell for producing red blood cells into which the expression cassette or the recombinant vector is introduced.

The erythroid induction differentiation induction may be used interchangeably with ‘red blood cell production’.

In the present disclosure, the cell culture means in vitro cell growth in an artificial medium for research or medical treatment.

The culture of the present disclosure may be performed using any method known in the art as long as the object of the present disclosure can be achieved, and may be arbitrarily controlled by those skilled in the art as long as the shape and activity of the cell do not exhibit abnormalities.

That is, the culturing process of the present disclosure may be performed according to media and culture conditions known in the art. The culturing process may be easily adjusted and used by those skilled in the art according to a selected cell.

The culture may be performed using a stationary culture or suspension culture method. The stationary culture means culturing in a state where the culture is left in an incubator without agitating or shaking, and the suspension culture means culturing in a state where the cells are suspended without being attached to the bottom or side of a reactor through aeration or stirring. In addition, the reactor for stationary culture and the reactor for suspension culture may be the same as or different from each other.

In a specific embodiment of the present disclosure, the transformed cells for producing red blood cells may be 0.1×10⁵ to 20×10⁵ cells/ml, preferably 1×10⁵ to 2×10⁵ cells/ml, and most preferably 0.75×10⁵ cells/ml.

In a specific embodiment of the present disclosure, the erythroid differentiation is preferably differentiation-inducing and enucleating erythroid progenitor cells into red blood cells.

In the present disclosure, the differentiation induction refers to a change from a default cell type (genotype and/or phenotype) to a non-default cell type (genotype and/or phenotype). Accordingly, the “erythroid differentiation induction” refers to induction of division of hematopoietic stem cells into progeny cells (i.e., red blood cells) having characteristics that differ from those of hematopoietic stem cells, such as genotype (e.g., changes in gene expression determined by genetic analysis) and/or phenotype (e.g., changes in protein expression). In a broad sense, the induction of erythroid differentiation of the present disclosure means inducing a change from hematopoietic stem cells or erythroid cells to progeny cells (i.e., red blood cells) through endothelial-to-hematopoietic transition, hematopoiesis induction, or enucleation induction.

In the present disclosure, the enucleation refers to a process in which the nucleus of an erythroid progenitor cell gradually condenses during differentiation and the erythroid progenitor cell loses the condensed nucleus. After enucleation, the erythroid progenitor cells become reticulocytes, and ultimately become erythrocytes through a maturation process.

In a specific embodiment of the present disclosure, the recombinant vector includes a promoter that regulates the expression of a gene, and further includes treating an expression inducer after the erythroid differentiation step.

In a preferred embodiment of the present disclosure, the expression inducer may be doxycycline or tetracycline.

In a specific embodiment of the present disclosure, the erythroid differentiation step may include: (a) culturing an erythroid progenitor cell in a medium containing IL-3, a stem cell factor (SCF), and the transformed cell for producing red blood cells; (b) culturing an erythroid progenitor cell in a medium containing SCF and the transformed cell for producing red blood cells; and (c) culturing an erythroid progenitor cell in a medium containing the transformed cell for producing red blood cells.

In a specific embodiment of the present disclosure, the method may further include ‘confirming the expression of an erythroid differentiation marker or an enucleation marker’. This is a process of confirming whether the finally obtained cells are red blood cells, and the expression of erythroid differentiation markers (CD71, GPA (CD235a)) and an enucleation marker (DRAQ5) may be confirmed using analysis methods (e.g., flow cytometry, microscopic observation) known in the art.

The red blood cells differentiated and produced by the method according to the present disclosure may be used in the form of an artificial blood composition, etc. The artificial blood composition of the present disclosure may be transfused into a subject, and may contribute to solving problems such as blood shortage and transfusion side effects. In addition, the produced red blood cells and the artificial blood composition may be effectively used in clinical fields related to blood; and in the field of treating diseases related to red blood cell reduction.

Duplicated contents will be omitted in consideration of the complexity of the present specification, and terms not defined otherwise herein have the meanings commonly used in the art to which the present disclosure pertains.

### [Modes]

Hereinafter, the present disclosure will be described in more detail through Examples. These Examples are just illustrative of the present disclosure, and it will be apparent to those skilled in the art that the scope of the present disclosure is not to be construed as being limited by these Examples.

### EXPERIMENTAL EXAMPLES

### Experimental Example 1. Culture of Human Pluripotent Stem Cells (hPSCs)

CMC-hiPSC-011 cells, obtained from the National Stem Cell Bank, were maintained in an Essential 8 (E8) medium (Gibco A1517001) of iMatrix-511 (Matrixome, 892 021) and fed daily. hPSCs were isolated into clumps using ReLeSR (Stemcell Tech., 05873), subcultured, and then seeded again in the E8 medium containing Y-27632 (2 μM; Stemcell Tech, 72307), a p160-ROCK (Rho-associated coiled-coil kinase) inhibitor.

### Experimental Example 2. Culture of UT7 Cells

UT7 cells were cultured in a Gibco MEM α medium (Thermo Fisher Scientific #12571063) under 37 °C and 5% CO₂ conditions. The Gibco MEM α medium contained 20% FBS (Gibco, Thermo Fisher Scientific #16000044) and 100 g/ml penicillin-streptomycin (WELGENE #LS 202-02), and was supplemented with 2 U/ml erythropoietin (EPO) (PeproTech, 100-64). In a control group, cells were cultured in a Gibco MEM α medium without EPO. The UT7 cells were subcultured every 2-3 days at a density of 2.5×10⁵ cells/ml.

### Experimental Example 3. Construction of MB-EPO Cell Lines Expressing Inducible EPO

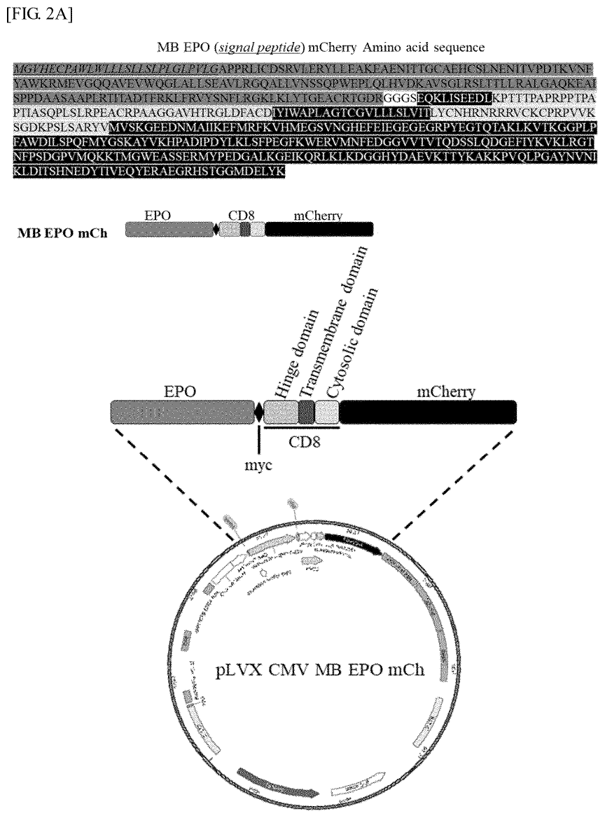

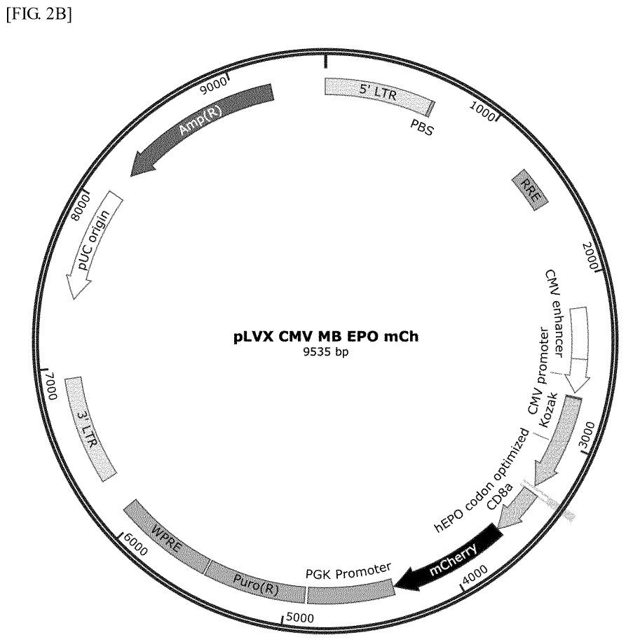

In Example, MB-EPO cells expressing inducible EPO were prepared. To prepare an MB-EPO cell line, 2×10⁶ cells of the human CMC11 iPSC cell line or UT7 cell line were prepared, and the prepared cells were introduced with a knock-in vector (pL VX CMV MB EPO mCh) (5 μg) and AAVS1-targeting TALEN (2 μg) of FIG. 2 by electroporation (Amaxa 4D Nucleofector system (Lonza)).

A cleavage map of the knock-in vector was shown in FIGS. 2A and 2B. The cells introduced with the knock-in vector expressed an ‘MB-EPO mCherry peptide’ represented by an amino acid sequence of SEQ ID NO: 1, and the peptide was encoded by a nucleotide sequence of SEQ ID NO: 11. Specifically, in the MB-EPO mCherry peptide, EPO (SEQ ID NO: 2), a linker (SEQ ID NO: 3), myc (SEQ ID NO: 4), CD8 (SEQ ID NO: 5), and mCherry (SEQ ID NO: 9) were sequentially and operably linked. The CD8 included a hinge domain (SEQ ID NO: 6), a transmembrane domain (SEQ ID NO: 7), and a cytosolic domain (SEQ ID NO: 8). The EPO, linker, myc, CD8 and mCherry were encoded by nucleotide sequences of SEQ ID NOs: 12, 13, 14, 15 and 19, respectively. In addition, the hindge domain, transmembrane domain, and cytosolic domain were encoded by nucleotide sequences of SEQ ID NOs: 16 to 18, respectively.

Thereafter, an E8 medium supplemented with Y-27632 (10 μM) was dispensed into a 6-well plate, and a serial cell dilution was seeded into a 6-well plate. Clones were selected using antibiotics, and the selected clones were expanded and screened by Dox treatment and Cas9 staining. For genotyping, genomic DNA was extracted using a DNeasy Blood & Tissue Kit. Junction PCR was performed using the extracted genomic DNA as a template using KOD-Multi & Epi (Toyobo, KME-101).

### Experimental Example 4. Endothelial-to-Hematopoietic Transition (EHT) Step after Mesoderm Induction

Undifferentiated induced pluripotent stem cells (iPSCs) were maintained in an Essential 8 (E8) medium (Gibco A1517001) of iMatrix-511 (Matrixome, 892 021). The cells were isolated using TrypLE (Gibco 12563011) and cultured for 2 days in a mTeSR™3D medium (StemCell Technologies 3950) on an orbital shaker set at 70 rpm according to the manufacturer's instructions. Subsequently, pluripotent spheroids were transferred to an STEMdiff™ APEL™2 medium containing 10 μM CHIR-99021 (Selleckchem), 50 ng/ml BMP4 (Miltenyi), and 50 ng/ml VEGF-165 (Miltenyi) and cultured for 2 days. Thereafter, CHIR was removed and then the cells were cultured for 2 days. Finally, the cells were cultured for 2 days in the presence of 50 ng/ml BMP4 (Miltenyi), 50 ng/ml VEGF-165 (Miltenyi), and 50 ng/ml SCF (Miltenyi). After mesoderm induction for 6 days was completed, 20 embryoid bodies (EBs) were transferred to a 35 mm gelatin-coated dish (Sigma G1393) and cultured with a medium for 4 days. The medium contained STEMdiff APEL2 medium (STEMCELL Technologies), 5% PFHM-II protein-free hybridoma medium (Thermo Fisher Scientific), 10 ng/ml BMP4, 100 ng/ml SCF, 100 ng/ml FLT3-L (Miltenyi), 50 ng/ml TPO (Miltenyi), 50 ng/ml VEGF-165, 25 ng/ml IL-3 (PeproTech), 25 ng/ml IL-6 (PeproTech), 10 ng/ml IGF-II (PeproTech) and 10 ng/ml FGF-2 (R&D Systems). Thereafter, the medium was replaced with STEMdiff APEL2 medium (STEMCELL Technologies), 5% PFHM-II protein-free hybridoma medium (Thermo Fisher Scientific), 100 ng/ml SCF, 100 ng/ml FLT3-L, 50 ng/ml TPO, 25 ng/ml IL-3, and 25 ng/ml IL-6.

### Experimental Example 5. Hematopoietic Induction

After 8 days of EHT, hematopoiesis of cells was induced in a hematopoietic induction medium for 7 days until erythroid progenitor cells were induced. The hematopoietic induction medium contained STEMdiff APEL2 medium (STEMCELL Technologies), 5% PFHM-II protein-free hybridoma medium (Thermo Fisher Scientific), 5 ng/ml IL-3, 100 ng/ml SCF, 3 U/ml EPO (PeproTech), and 1 μM dexamethasone (Stem Cell Technologies). For erythroid differentiation, a hematopoietic-induced cell suspension (i.e., erythroid progenitor cells) was harvested.

### Experimental Example 6. Erythroid Differentiation

According to a three-step erythropoiesis protocol, erythroid differentiation of the erythroid progenitor cells (i.e., hematopoietic-induced cells) harvested in Example 5 was induced. To induce the erythroid differentiation, the erythroid progenitor cells were cultured for 13 days in a StemSpan™ SFEM II medium supplemented with 10% AB serum (Innovative Research ISERAB) and 330 mg/mL transferrin (R&D Systems). Specifically, differentiation was induced from day 0 to day 8 using a medium containing 5 ng/ml IL-3, 100 ng/ml SCF, and 1.5×10⁵ cells/2 ml of MB-EPO KI cells. In addition, differentiation was induced from day 8 to day 11 by replacing the medium with a medium containing 100 ng/ml SCF and 1.5×10⁵ cells/2 ml of MB-EPO KI cells. Finally, differentiation was induced from day 8 to day 13 by replacing the medium with a medium containing 1.5×10⁵ cells/2 ml of MB-EPO KI cells. The culture period may be extended to 18 days as needed. Hematopoiesis and erythroid differentiation were determined by flow cytometry and microscopic evaluation at specified intervals.

Meanwhile, in a control group, differentiation was induced from day 0 to day 8 using a medium containing 5 ng/ml IL-3, 100 ng/ml SCF, and 3 U/ml EPO. In addition, differentiation was induced from day 8 to day 11 by replacing the medium with a medium containing 100 ng/ml SCF and 3 U/ml EPO. Finally, differentiation was induced from day 8 to day 13 by replacing the medium with a medium containing 3 U/ml EPO.

Recombinant human EPO, IL-3, and SCF used in the experiment were purchased from PEPROTECH.

### Experimental Example 7. Cell Counting

The number of surviving cells was counted by trypan blue staining. Hematopoietic cells and red blood cells were harvested, centrifuged, and then the supernatant was removed, and the cells were suspended in a medium and washed. The suspended cells were stained at 1:1 with a trypan blue solution and then the cell number was measured using a cell counter (LunaII cell counter, Logos Biosystems).

### Experimental Example 8. Flow Cytometry

Cells were washed with PBS, suspended in PBS/5% fetal bovine serum, and reacted with 10 μl of each monoclonal antibody at 4 °C for 30 minutes. To confirm MB-EPO KI, the cells were labeled with Myc-tag-Alexa 488 (Cell Signaling), and IgG2-Alexa 488 (Cell Signaling) was used for isotypic control staining. To analyze the expression of surface protein markers of in vitro differentiated red blood cells, the cells were labeled with the following anti-human antibodies. CD71-PE (BD Pharmingen), glycophorin A (GPA)-FITC (BD Pharmingen), and immunoglobulins G2 IgG2-FITC and IgG2-PE (BD Pharmingen) were used for isotypic control staining. The cells were washed twice with PBS/5% FBS, resuspended in 300 μl formaldehyde (2%), and then measured using a BD FACSAria II (BD Bioscience). Data from flow cytometry were analyzed using FlowJo™ Software (BD Bioscience).

### Experimental Example 9. Microscopic Evaluation

Cells were stained with trypan blue, and the stained cells were observed using an inverted microscope (Ts2-FL, Nikon). In addition, for observation of red blood cells, the cells were stained with Wright-Giemsa and the stained cells were observed under a light microscope.

### EXAMPLES

### Example 1. Cell Viability Analysis of UT7-MB-EPO Cells According to Presence of EPO

### 1-1. Cell Growth Analysis of UT7 Cells According to Presence of EPO

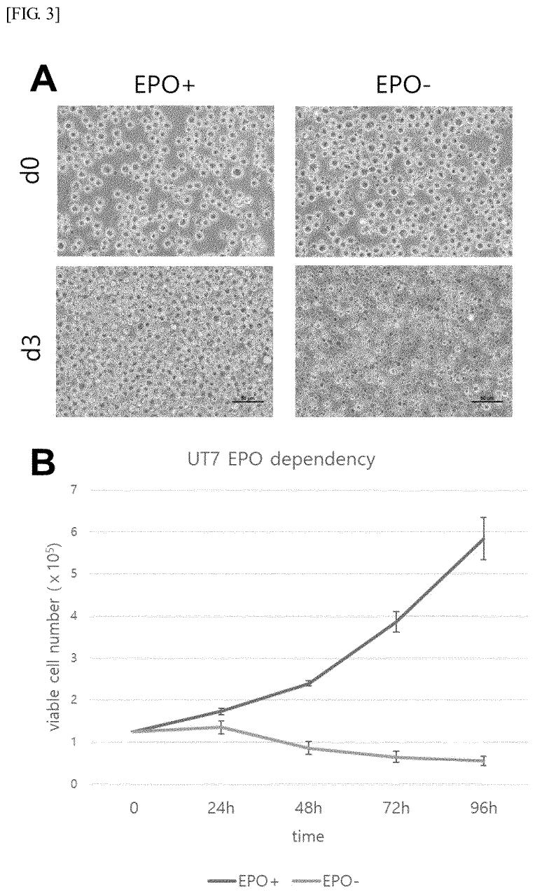

Cell growth of UT7 cells according to the presence of EPO was analyzed. Specifically, the UT7 cells were cultured using a medium containing EPO as in Experimental Example 2 (EPO+ group). A control group was cultured in a medium without EPO (EPO group). The cells were evaluated under microscopy and counted every 24 hours from the start of culture. Cell viability was calculated after cell counting. The results of microscopic evaluation and cell growth according to the presence of EPO were shown in FIGS. 3A and 3B, respectively.

As shown in FIG. 3A, the EPO+ group showed normal cell morphology even on day 3 (72 hours after culture). On the other hand, the EPO-group (control group) showed abnormal cell morphology and a large number of cells were dead.

As shown in FIG. 3B, significant cell proliferation was observed in the EPO+ group. In contrast, no significant proliferation was observed in the EPO-group.

### 1-2. Construction of UT7-MB-EPO Cells

UT7 cells were cultured using the method of Experimental Example 2, and the cells were introduced with lentiviral particles containing a gene encoding an MB-EPO mCherry peptide as in Experimental Example 3. The UT7 cells introduced with MB-EPO mCherry peptide were named ‘UT7-MB-EPO cells’. In the UT7-MB-EPO cell, EPO was linked to a cell surface antigen CD8, and the CD8 was bound to the cell membrane. Accordingly, EPO bound to the UT7-MB-EPO cell membrane continuously affected an EPO receptor of the cell, and as a result, EPO-dependent gene expression was induced (FIG. 1).

In a control group, cells were introduced with a gene encoding GFP instead of the MB-EPO mCherry peptide and were named ‘UT-GFP cells’.

### 1-3. Cell Growth Analysis of UT7-MB-EPO Cells According to Presence of EPO

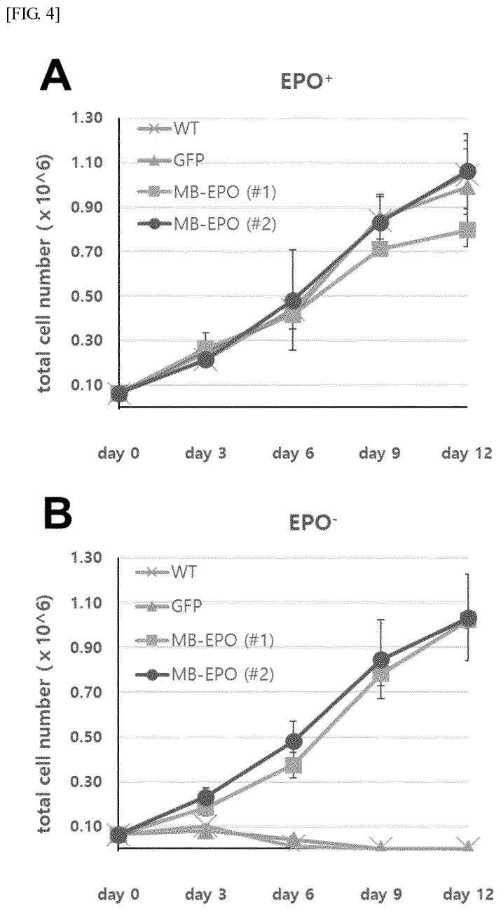

Cell growth of UT7-MB-EPO cells (MB-EPO) constructed in Example 1-2 was analyzed according to the presence of EPO. The cell number was counted every 3 days of culture and analyzed for 12 days. Control groups were wild-type UT7 cells (WT); and UT-GFP cells (GFP) into which a GFP gene has been introduced. The results of analyzing cell growth according to the presence and absence of EPO were shown in FIGS. 4A and 4B, respectively.

As shown in FIG. 4A, when the cells were cultured in the presence of EPO, the cell growth of the experimental group (MB-EPO) and the control groups (WT, GFP) showed similar trends.

As shown in FIG. 4B, the control groups (WT, GFP) died when cultured in the absence of EPO. In contrast, MB-EPO showed similar cell growth as in the presence of EPO (FIG. 4A).

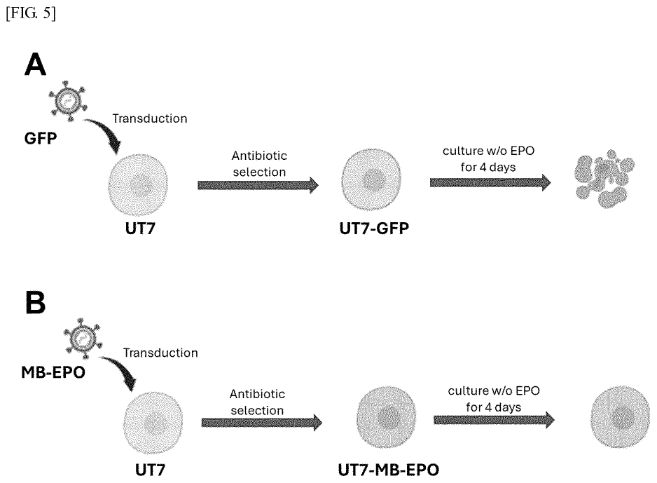

Based on the results, the cell growth depending on whether a gene encoding the MB-EPO mCherry peptide was introduced was shown in FIG. 5. Specifically, GFP-transduced UT7 cells died in the absence of EPO (FIG. 5A). In contrast, it was confirmed that in MB-EPO, cell growth was performed even in the absence of EPO supplied to the medium. It is considered because EPO bound to the UT7-MB-EPO cell membrane continuously affects a cell receptor, and as a result, EPO-dependent gene expression is induced without adding new EPO during the culture process.

### Example 2. Construction of MB-EPO-KI hiPSCs for In Vitro Erythropoiesis and Erythropoiesis

2-1. Construction of MB-EPO-KI hiPSCs

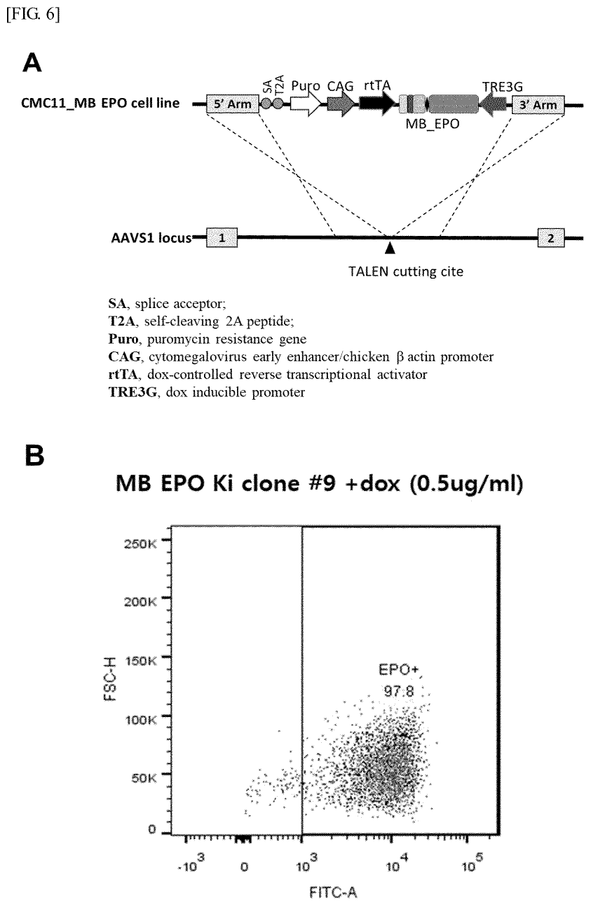

MB-EPO-KI hiPSCs were constructed in which the expression of MB-EPO introduced into the cell was induced by doxycycline (dox). A TALEN-mediated targeting strategy of an AAVS1 locus for constructing CMC11-MB-EPO cells was shown in FIG. 6A. An AAVS1 donor vector contained an MB-EPO gene cassette that may be controlled by a dox-inducible TRE3G promoter. The CMC11 cells were introduced with the vector in FIG. 6A, which were named ‘MB-EPO-KI hiPSCs’. The constructed MB-EPO-KI hiPSCs expressed MB-EPO in the presence of dox.

The expression of MB-EPO in the constructed MB-EPO-KI hiPSCs was verified. Specifically, the constructed MB-EPO-KI hiPSCs were cultured in a doxycycline-containing culture medium to induce the expression of MB-EPO. Thereafter, the MB-EPO expressing cells were identified through flow cytometry. The flow cytometry results were illustrated in FIG. 6B.

As shown in FIG. 6B, it was confirmed that the MB-EPO expressing cells were 97.8% in the cell population.

2-2. In Vitro Erythropoiesis of MB-EPO-KI hiPSCs

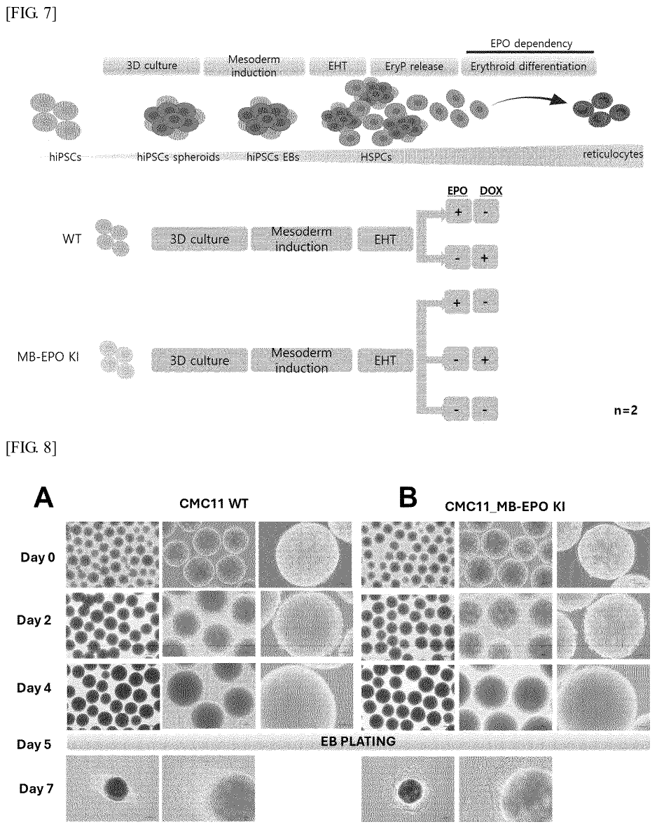

hiPSCs were 3D-cultured to form hiPSC spheroids, and then red blood cells were produced through mesoderm induction and endothelial-to-hematopoietic transition (FIG. 7).

In Example, in vitro erythropoiesis was performed using MB-EPO-KI hiPSCs constructed in Example 2-1, as shown in FIG. 7. A control group was wild-type hiPSCs (WT).

In experiments to be described below, MB-EPO-KI hiPSCs and the control group WT were divided into five groups according to the presence of EPO and doxycycline, and the morphology of cells at each differentiation stage was analyzed under a microscope.

Morphological Analysis of MB-EPO-KI hiPSCs During Mesoderm Induction

Morphological analysis of a control group CMC11 WT and MB-EPO-KI hiPSCs was performed during mesoderm induction. The morphological analysis results of the control group CMC11 WT and MB-EPO-KI hiPSCs were shown in FIGS. 8A and 8B, respectively.

As shown in FIGS. 8A and 8B, both the control group CMC11 WT and MB-EPO-KI hiPSCs normally formed embryoid bodies (EBs), and it was confirmed through a microscope that the shapes thereof were maintained even after EB plating.

Morphological Analysis of MB-EPO-KI hiPSCs in Endothelial-to-Hematopoietic Transition (EHT)

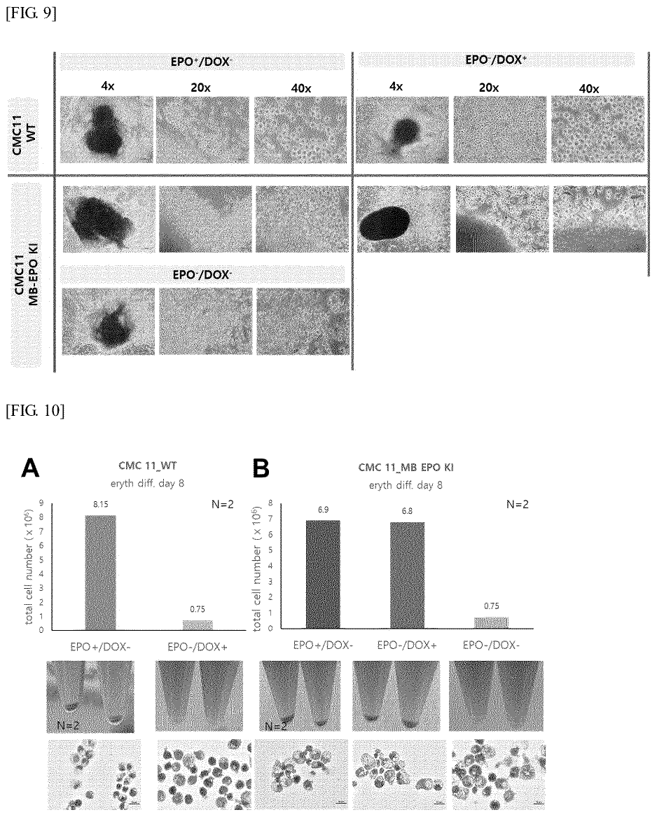

Morphological analysis of a control group WT and MB-EPO-KI hiPSCs was performed after mesoderm induction. Results of morphological analysis of the control group WT and MB-EPO-KI hiPSCs depending on the presence of EPO and doxycycline during endothelial-to-hematopoietic transition were shown in FIG. 9.

As shown in FIG. 9, it was confirmed through microscopy that hematopoietic single suspension cells were formed during endothelial-to-hematopoietic transition in all experimental groups.

### Evaluation of Erythroid Differentiation

The erythroid differentiation process was an EPO-dependent process, and erythroid differentiation was induced by culturing cells in an EPO-containing medium as in Experimental Example 6 above. After induction of erythroid differentiation, erythroid differentiation was evaluated through cell counting, visual observation, and microscopic observation. The results of evaluating erythroid differentiation of a control group WT and MB-EPO-KI hiPSCs were shown in FIGS. 10A and 10B, respectively.

As shown in FIG. 10A, it was confirmed that the control group WT produced red blood cells only in the presence of EPO.

As shown in FIG. 10B, it was confirmed that MB-EPO-KI hiPSCs produced red blood cells under EPO+/DOX− and EPO−/DOX+ conditions. In contrast, MB-EPO-KI hiPSCs did not produce red blood cells under EPO−/DOX− conditions. The results showed that MB-EPO-KI hiPSCs induced MB-EPO expression in the presence of doxycycline, thereby producing red blood cells even without EPO.

In summary, the present inventors have constructed an MB-EPO cell line expressing inducible EPO. In the MB-EPO cell line according to the present disclosure, EPO is linked to a cell surface antigen CD8, and the CD8 is bound to the cell membrane. Therefore, EPO bound to the cell membrane continuously affects a cell receptor to induce the expression of EPO-dependent genes (i.e., inducing the expression of genes related to erythroid differentiation). It is meant that when the MB-EPO cell line expressing the inducible EPO of the present disclosure is used, erythroid differentiation may be induced even without adding EPO, and thus the cell line may be variously used in fields related to artificial blood.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

1. An expression cassette for producing red blood cells comprising:
an erythropoietin (EPO) gene represented by a nucleotide sequence of SEQ ID NO: 12; and
a gene encoding a transmembrane domain.

2. The expression cassette for producing red blood cells of claim 1, wherein the transmembrane domain is at least one selected from the group consisting of a receptor, a ligand, an immunoglobulin, and a glycophorin.

3. The expression cassette for producing red blood cells of claim 2, wherein the transmembrane domain is at least one selected from the group consisting of CD8, CD4, CD3, CD3y, CD38, CD3S, CD28, CD137, Fcely, T cell receptor, nicotinic acetylcholine receptor, and gamma-aminobutyric acid receptor.

4. The expression cassette for producing red blood cells of claim 1, further comprising:
a linker.

5. The expression cassette for producing red blood cells of claim 4, wherein an erythropoietin gene, the linker, and a gene encoding the transmembrane domain are sequentially operably linked.

6. A recombinant vector for producing red blood cells comprising: an expression cassette for producing red blood cells comprising an erythropoietin (EPO) gene represented by a nucleotide sequence of SEQ ID NO: 12; and a gene encoding a transmembrane domain.

7. The recombinant vector of claim 6, wherein the recombinant vector includes a promoter that regulates an expression of the gene.

8. The recombinant vector of claim 7, wherein the promoter is a tetracycline response element (TRE) promoter.

9. A transformed cell for producing red blood cells comprising:
an expression cassette for producing red blood cells comprising an erythropoietin (EPO) gene represented by a nucleotide sequence of SEQ ID NO: 12; and a gene encoding a transmembrane domain; or
a recombinant vector comprising the expression cassette.

10. The transformed cell for producing red blood cells of claim 9, wherein the transformed cell is a stem cell.

11. The transformed cell for producing red blood cells of claim 10, wherein the stem cell is at least one selected from the group consisting of an induced pluripotent stem cell (iPSC), a hematopoietic stem cell (HSC), and a mesenchymal stem cell (MSC).

12. The transformed cell for producing red blood cells of claim 11, wherein erythropoietin is bound to the transmembrane domain.

13. The transformed cell for producing red blood cells of claim 12, wherein the transmembrane domain is bound to a cell membrane.

14. (canceled)

15. (canceled)

16. (canceled)

17. The transformed cell for producing red blood cells of claim 9, wherein transformed cell activating cell expresses erythropoietin bound to a transmembrane domain located on a cell membrane.

18. (canceled)

19. A method for inducing in vitro erythroid differentiation, comprising an erythroid differentiation step of culturing a transformed cell for producing red blood cells into which the expression cassette according to claim 1; and an erythroid progenitor cell.

20. (canceled)

21. (canceled)

22. (canceled)

23. (canceled)

24. (canceled)


SEQUENCE LISTING (20 sequences)

SEQ ID NO: 1 - protein, 543 aa, synthetic construct
  source 1..543: note = MB-EPO mCherry
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGGGSEQKLISEEDLKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 2 - protein, 193 aa, Homo sapiens
  source 1..193: note = EPO
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 3 - protein, 4 aa, synthetic construct
  source 1..4: note = linker
GGGS

SEQ ID NO: 4 - protein, 10 aa, Homo sapiens
  source 1..10: note = myc
EQKLISEEDL

SEQ ID NO: 5 - protein, 100 aa, Homo sapiens
  source 1..100: note = CD8
KPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 6 - protein, 47 aa, Homo sapiens
  source 1..47: note = hinge domain
KPTTTPAPRPPTPAPTIASQPLSLRPEACRPAAGGAVHTRGLDFACD

SEQ ID NO: 7 - protein, 21 aa, Homo sapiens
  source 1..21: note = transmembrane domain
IYIWAPLAGTCGVLLLSLVIT

SEQ ID NO: 8 - protein, 32 aa, Homo sapiens
  source 1..32: note = cytosolic domain
LYCNHRNRRRVCKCPRPVVKSGDKPSLSARYV

SEQ ID NO: 9 - protein, 236 aa, synthetic construct
  source 1..236: note = mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 10 - protein, 239 aa, synthetic construct
  source 1..239: note = GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 11 - DNA, 1632 bp, synthetic construct
  source 1..1632: mol_type = other DNA; note = MB-EPO mCherry
atgggtgttcacgagtgccccgcgtggctgtggcttctcctttcacttttgagcctgccgcttggattgccagtgttgggggcaccaccgcgattgatctgcgatagtcgcgtacttgaacgatacctgctggaagctaaagaggccgaaaacataaccacgggctgcgccgagcattgttcattgaacgagaacataactgtcccggataccaaagtgaacttctatgcctggaaacgaatggaggttgggcagcaggcagtcgaggtttggcaaggtctggctctcctgtcagaagcggttctcaggggacaagcattgcttgtaaatagctcacagccctgggagcccctccaactgcacgtcgacaaggcggtcagtggccttcgctccctcacaactctgctcagagcccttggtgctcaaaaggaagcaatctccccgcccgatgctgcatcagcggcacctctgcgaacaattacggctgacacattccgcaagttgtttcgggtctattctaactttttgcgaggtaaacttaaactgtacacgggcgaagcatgtcgaacgggagacagaggcggaggaagcgagcagaagctgatctccgaggaagatctgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 12 - DNA, 579 bp, Homo sapiens
  source 1..579: mol_type = genomic DNA; note = EPO
atgggtgttcacgagtgccccgcgtggctgtggcttctcctttcacttttgagcctgccgcttggattgccagtgttgggggcaccaccgcgattgatctgcgatagtcgcgtacttgaacgatacctgctggaagctaaagaggccgaaaacataaccacgggctgcgccgagcattgttcattgaacgagaacataactgtcccggataccaaagtgaacttctatgcctggaaacgaatggaggttgggcagcaggcagtcgaggtttggcaaggtctggctctcctgtcagaagcggttctcaggggacaagcattgcttgtaaatagctcacagccctgggagcccctccaactgcacgtcgacaaggcggtcagtggccttcgctccctcacaactctgctcagagcccttggtgctcaaaaggaagcaatctccccgcccgatgctgcatcagcggcacctctgcgaacaattacggctgacacattccgcaagttgtttcgggtctattctaactttttgcgaggtaaacttaaactgtacacgggcgaagcatgtcgaacgggagacaga

SEQ ID NO: 13 - DNA, 12 bp, synthetic construct
  source 1..12: mol_type = other DNA; note = linker
ggcggaggaagc

SEQ ID NO: 14 - DNA, 30 bp, Homo sapiens
  source 1..30: mol_type = genomic DNA; note = myc
gagcagaagctgatctccgaggaagatctg

SEQ ID NO: 15 - DNA, 303 bp, Homo sapiens
  source 1..303: mol_type = genomic DNA; note = CD8
aagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 16 - DNA, 141 bp, Homo sapiens
  source 1..141: mol_type = genomic DNA; note = hinge domain
aagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 17 - DNA, 63 bp, Homo sapiens
  source 1..63: mol_type = genomic DNA; note = transmembrane domain
atctacatctgggcgcccttggccgggacttgtggggtccttctcctgtcactggttatcacc

SEQ ID NO: 18 - DNA, 96 bp, Homo sapiens
  source 1..96: mol_type = genomic DNA; note = cytosolic domain
ctttactgcaaccacaggaaccgaagacgtgtttgcaaatgtccccggcctgtggtcaaatcgggagacaagcccagcctttcggcgagatacgtc

SEQ ID NO: 19 - DNA, 711 bp, synthetic construct
  source 1..711: mol_type = other DNA; note = mCherry
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 20 - DNA, 720 bp, synthetic construct
  source 1..720: mol_type = other DNA; note = GFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa